(19) United States
(12) Patent Application Publication
(10) Pub. No.: US 2011/0200625 A1
(43) Pub. Date: Aug. 18, 2011

(54) PHARMACEUTICAL COMPOSITION

(76) Inventor: Shaiw Wen Liu, Honeybrook, PA (US)

(21) Appl. No.: 13/026,870

(22) Filed: Feb. 14, 2011

Related U.S. Application Data
(60) Provisional application No. 61/304,122, filed on Feb. 12, 2010.

(51) Int. Cl. A61K 31/436; A61K 39/00; A61K 31/164; A61K 38/13; A61P 9/00; A61P 35/00; A61P 17/02; A61P 17/00; A61P 37/06

(52) U.S. Cl. 424/184.1; 514/291; 514/616; 514/13.3

(57) ABSTRACT

A nonirritant, thickened pharmaceutical composition comprising a drug which is normally solid and which has anti-angiogenesis properties and/or immunosuppressant properties, a normally solid dermal penetration-facilitator for the drug, a solvent which is capable of dissolving the drug and the facilitator; and a thickening agent.

### CROSS-REFERENCE TO RELATED APPLICATION

This application claims the benefit of the filing date of Provisional Application No. 61/304,122, filed Feb. 12, 2010.

The present invention relates to a pharmaceutical composition which upon application to the surface of the skin is effective in blocking the formation of new blood vessels. More particularly, the invention relates to a pharmaceutical composition which contains an anti-angiogenesis drug, that is, a drug which blocks the formation of new blood vessels, and to the use of the composition.

The present invention will be described initially in connection with its use in the treatment of a port wine stain birthmark (hereafter “PWS birthmark”). However, the invention can be used to treat other undesirable bodily conditions, as explained below.

PWS birthmark is a congenital, progressive vascular malformation of skin involving post-capillary venules (small veins); it occurs in an estimated 4 children per 1,000 live births. Approximately 1,200,000 individuals in the United States and twenty-six million people worldwide have PWS birthmarks. Since most of the malformations occur on the face, PWS birthmarks pose a clinically significant problem in the majority of patients. Personality development is influenced adversely in virtually all patients by the negative reaction of others to a “marked’ person. A PWS birthmark is initially a flat red spot, but lesions tend to darken progressively to purple and, by middle age, often become raised as a result of the development of vascular nodules. Hypertrophy of underlying soft tissue disfigures further the facial feature of an afflicted person.

It is known to treat a PWS birthmark with a laser for the purpose of blanching the birthmark. Laser treatment is based on photocoagulation of the subsurface-targeted blood vessels without inducing thermal damage in the normal overlying epidermis. Treatment which involves the use of a pulsed dye laser (PDL) is approved by the Food and Drug Administration for the treatment of a PWS birthmark. Light which is emitted by the use of PDL is absorbed preferentially by hemoglobin (major chromophore in blood) in the PWS birthmark blood vessels where, after being converted to heat, causes thermal damage and thrombosis. Through selective photothermolysis, PDL exposure destroys subsurface PWS blood vessels in human skin. PDL treatment of a PWS birthmark induces a purpuric response in human skin, which is associated with hypoxia, inflammation and edema in the upper layers of the skin. Inflammatory cells migrate into the area secreting cytokines, which are potent up-regulators of hypoxia-inducible factor 1-α (HIF1-α) and vascular endothelial growth factor (VEGF). Although clinically resulting in intense purpura, with histological documentation of vascular wall necrosis in vessels located 1-2 mm below the skin surface, the laser-induced wound-healing response to PDL treatment results often in reformation of the “PWS” blood vessels. In simple terms, the laser does what it is supposed to do, namely, cause blood vessel wall necrosis. Regrettably, the body does also what it is supposed to do, namely, repair the laser-induced damage.

It is known also to use cryogen spray cooling (CSC) prior to treatment with laser irradiation; CSC has been demonstrated to prevent skin surface textural changes such as scarring, atrophy, induration or dyspigmentation. In the absence of CSC, epidermal necrosis and subsequent skin surface textural changes are observed when the treatment is administered with equivalent laser irradiation parameters.

As mentioned briefly above, the goal of the aforementioned treatment is to blanch the color of the PWS birthmark, that is, to lighten the wine color of the birthmark to a readily apparent degree; however, the degree of blanching which is observed following PDL treatment remains variable and unpredictable. It is often very difficult to estimate how many treatments will be required to achieve the desired level of improvement. Moreover, due to the reformation of PWS blood vessels, there have been reports of PWS becoming darker and redder after PDL treatment.

If the ultimate standard required is complete lesion blanching, the average success rate is below 10%, even after undergoing numerous PDL treatments. Moreover, less than 50% of patients achieve 50% fading of their PWS in response to PDL therapy. Accordingly, an object of the present invention is to maintain the positive “blanched” effect obtained by use of the aforementioned treatment of a PWS birthmark.

### SUMMARY OF THE INVENTION

Broadly stated, the present invention provides a nonirritant pharmaceutical composition comprising an anti-angiogenesis and/or immunosuppressant drug and a dermal penetration-facilitator for the drug.

One embodiment of the present invention comprises a nonirritant composition which can be applied to the surface of the skin of a recipient for the treatment of a PWS birthmark or other undesirable bodily condition and which contains: (A) a drug having anti-angiogenesis and/or immunosuppressant properties; and (B) a dermal penetration facilitator for the drug. In the preferred treatment of a PWS birthmark, the composition is applied to the skin after the birthmark is blanched, for example, by use of the aforementioned laser-based treatments.

Another embodiment of the present invention is the provision of a nonirritant pharmaceutical composition which comprises: (A) a drug having anti-angiogenesis and/or immunosuppressant properties and which is, at room temperature, normally a solid that is insoluble in water and conventional organic solvents used in pharmaceutical compositions, but which is present in the composition in dissolved form and in a pharmaceutically effective amount; (B) a solvent which is capable of dissolving the drug and which is present in the composition in an amount sufficient to dissolve the drug; and (C) a dermal penetration-facilitator (also in dissolved form) for the dissolved drug. The penetration-facilitator is present in the composition in an amount which, upon topical application of the composition to the outer surface of the skin of a recipient, facilitates delivery of the dissolved drug to the dermal layer of the skin; and this is accomplished with minimal or no systemic delivery of the dissolved drug into the blood stream of the recipient. The preferred drug for use in such composition is rapamycin and the particularly preferred penetration-facilitator is pentadecalactone; also, it is preferred that the composition be in a thickened form.

### DETAILED DESCRIPTION OF THE INVENTION

As mentioned above, one embodiment of the present invention is a pharmaceutical composition which comprises a drug having anti-angiogenesis and/or immunosuppressant properties, a solvent therefor, and a dermal penetration-facilitator for the dissolved drug. The following is a description of each of these constituents and also other constituents that may be included in the composition.

The drug for use in the present invention is a compound which prevents revascularization (blood vessel formation). It is known to use such drugs to treat various types of undesirable bodily conditions which are associated with blood-vessel formation, as is known in the art.

Examples of drugs which have anti-angiogenesis and/or immunosuppressant properties include rapamycin (also known as sirolimus), tacrolimus, everolimus, gusperimus, pimecrolimus, tesirolimus, cyclosporine, and temsirolimus. Either one or two or more of such compounds can be used in the composition. Typically, compounds of this class of drugs are insoluble in water and common pharmaceutically acceptable organic solvents, with some of the drugs being highly insoluble in water and the organic solvents.

For the purpose of treating a PWS birthmark, the preferred drug is rapamycin. Rapamycin is a macrocyclic fermentation product of the microorganism Stroptomyces hygroscopis, an actinomycete isolated originally from a soil sample from Rapa Nui (Easter Island). Rapamycin was investigated initially as an antifungal and antitumor agent. Over the past 15 years, rapamycin has been used for immunosuppression in renal translplantation patients because of its unique lack of end-organ toxicity and ability to synergize with other agents without overlapping side effects. In addition to being an immunosuppressant, rapamycin inhibits also angiogenesis by multiple mechanisms. For example, rapamycin inhibits the proliferation of vascular endothelial cells driven by vascular endothelial growth factor (VEGF). Rapamycin suppresses also the induction of hypoxia-inducible factor 1-alpha (HIF1-α), which is a transcriptional regulator of VEGF expression. Therefore, rapamycin inhibits vascular endothelial cell proliferation by both decreasing VEGF and by inhibiting the mitogenesis of vascular endothelial cells in response to VEGF. In addition, rapamycin inhibits also vascular smooth muscle cell proliferation and migration.

With respect to the aforementioned vascular endothelial growth factor (VEGF), it is known that the mTOR receptor is involved in its functioning to promote the proliferation of vascular endothelial cells. It is believed that rapamycin and various other drugs for use in the present invention have an inhibitory effect on the mTOR receptor and this in turn leads to the inhibition of the normal proliferation of vascular endothelial cells that would otherwise be effected by VEGF.

The physical characteristics of rapamycin (and various of the other drugs for use in the present invention) make it difficult to formulate and use in a topical application at room temperature. It is a highly lipophilic, crystalline compound having a relatively high melting point in the range of 183-185° C., with very poor water-solubility at room temperature. The high lipophilicity requires lipophilic solvents for dissolution, for example, benzyl alcohol. The crystallinity of the compound predisposes it to crystallization from solution. Therefore, other solubilizing agents, including, for example, emulsifiers, surfactants, chelating agents and buffer are used to prevent or deter an aqueous-based formulation from depositing crystals at room temperature.

The rapamycin (or other drug for use in the present invention) should be present in the composition in a pharmaceutically effective amount. Such amount will vary depending on various factors, for example, the particular drug being used, the nature of the condition being treated, and the patient being treated. It is believed that, for most applications, the amount of the drug (or a mixture of two or more of the drugs) will comprise about 0.1 to about 10 wt. % of the composition. (Unless stated otherwise, the term “wt. %” when used herein means weight % based on the total weight of the composition.) In the treatment of a blanched PWS birthmark with a rapamycin-containing composition, it is recommended that the composition comprise about 0.1 to about 8 wt. %, of the rapamycin.

There is an advantage to including in the composition a drug, as described above, which is a compound that metabolizes to a compound that is itself a drug that has anti-angiogenesis and/or immunosuppressant properties. For example, the aforementioned compound temsirolimus is an ester analog of aforementioned serolimus (rapamycin); in use, it is metabolized to serolimus. It has been obversed that a composition of the present invention which includes temsirolimus has a relatively long positive effect in use. It is believed that this is attributed to the initial effect of the temsirolimus and the continuing effect of the “sirolimus” metabolite.

As mentioned above, the pharmaceutical composition of the present invention can be used for the treatment of other undesirable bodily conditions, for example, psoriasis, Kaposi's sarcoma, skin cancer and rosacea. Examples of drugs for treating such conditions include rapamycin and tacrolimus.

The dermal penetration-facilitator of the pharmaceutical composition of the present invention functions to promote the delivery of the drug to the dermal layer of the skin, with minimal or no systemic delivery into the blood stream of the recipient. While not wishing to be bound by any specific theory, it is believed that the dermal penetration-facilitator (for convenience, hereafter referred to also as “the facilitator”) of the present invention causes an increase in the fluidity of the overlapping cell membranes of the stratum corneum (outermost surface layer of the skin), thereby facilitating diffusion of the drug through the cells as well as around them. Thus, the facilitator acts like a solvent to actually enhance the solubility of drugs in the epidermal cell membrane lipids. The permeation-enhancing effect of the facilitator is predictable, temporary, and does not cause any long term change to the skin structure. A mixture of two or more of the facilitators can be used.

The preferred facilitator for use in the composition of the present invention is a member of the class of compounds which have become known in the art as the “Hsieh enhancers”, for example, as described in U.S. Pat. No. 5,023,252 to Hsieh. However, this patent describes the Hsieh enhancers as transdermal- or transmembrane-delivering compounds that enhance the delivery of the drug from the surface of the skin or nasal membrane into the blood stream. Accordingly, in accordance with the disclosure of the Hsieh patent, the Hsieh enhancers are used in applications in which they are combined with a drug, which to be effective, must be delivered systemically into the blood stream of the recipient. Examples of drugs which can be delivered systemically by such enhancers include the peptide insulin and the steroid fluorogestorone acetate.

In the present invention, the Hsieh enhancer is used in a different way, that is, as a penetration-facilitator for delivery of drug to the dermal layer of the skin without significant, if any, systemic absorption of the drug into the blood stream of the user. In order to accomplish this, the concentration of the dermal penetration-facilitator is adjusted, typically lowered, in a manner dependent on the specific drug used, in order to promote delivery of a pharmaceutically effective amount of the drug through the epidermis and to the dermal layer of the skin, but not to the bloodstream. Evidence of such delivery is that the drug, for example, rapamycin, has been observed to be present in the epidermis for prolonged periods of time after the composition containing the drug has been applied to the skin, for example, 72 hours after application.

Examples of Hsieh enhancers that can be used in the composition are 3-methylcyclopentadecanone (muscone), 9-cycloheptadecen-1-one (civetone), cyclohexadecanone, and cyclopentadecanone (normuscone). As mentioned above, the use of pentadecalactone is preferred.

It is believed that for most applications the effective amount of the penetration-facilitator of the present invention will fall in the range of about 0.1 to about 20 wt. % depending on the specific drug to be delivered and the condition to be treated. Preferably the amount of the penetration-facilitator will be about 2 to about 12 wt. %.

One or more liquids capable of dissolving the drug and the dermal penetration-facilitator may be used in the preparation of the pharmaceutical composition of the present invention, provided that its properties are suitable for a pharmaceutical use and the liquid is nonirritant. Indeed, the composition of the present invention is a nonirritant composition and, accordingly, the composition is formulated from ingredients and amounts thereof which impart to the composition nonirritant properties. The term “nonirritant composition” means that, upon application of the composition to the outer surface of skin (epidermis) whose blood vessels have been photocoagulated, the composition does not cause the recipient thereof to experience discomfort to a degree such that the recipient refrains from use of the composition as prescribed. The discomfort can manifest itself in various ways, for example, feelings of sharp pain, dull soreness, or of burning or stinging sensations. Light which is emitted by the use of PDL, as described above, can cause intense damages to the blood vessels of the epidermis, such damage being referred to herein also as “the wound”. The wound is typically very sensitive to contact by foreign substance. Accordingly, the composition of the present invention is “wound compatible”, that is, a nonirritant composition.

In view of the above, the solvent for use in the present invention is one which imparts to the composition nonirritant properties in the amount used; in addition, it is non-toxic, has a suitable vapor pressure, and is compatible with the other constituents of the composition. Ideally, it is effective in dissolving both the drug and the facilitator.

As mentioned above, compounds for use as drugs in the present invention are typically insoluble and/or indeed highly insoluble in water and other conventional organic solvents used in pharmaceutical compositions. The following are exemplary solvents which are known to be capable of dissolving, for example, rapamycin: dimethylsulfoxide (DMSO); dimethylformamide (DMF); and N-methylpyrrolidinone (NMP): however, they are considered toxic for human applications. In addition, it has been observed that DMSO is a transdermal carrier that can cause dissolved drugs to move directly into the blood stream from a topical application. The preferred solvent for use in the composition is benzyl alcohol. Examples of other solvents that can be used are: ethoxydiglycol, ethanol, and polysorbate 80. In a particularly preferred form, a single solvent is used, that is, benzyl alcohol.

The solvent(s) is present in the composition in an amount sufficient to dissolve the drug comprising the composition. The amount of the solvent(s) will depend on various factors including, for example, the particular solvent being used and the particular drug being used. For most applications, it is believed that the amount of solvent comprising the composition will be about 0.5 to about 30 wt. %.

The composition can be in any suitable form for topical application to the skin, for example, in the form of a cream or lotion. Also, a skin patch which contains the composition can be used to apply it.

In a preferred embodiment, the composition is in a thickened form such that it can be applied topically to the outer surface of the skin. In such form, the composition has a viscosity such that it is capable of being spread readily onto the surface of the skin and has sufficient cohesiveness to substantially retain its shape and presence on the skin after application. The term “ointment” is used herein to refer to preferred topical forms of the composition.

Any compound(s) which is suitable for use in a pharmaceutical composition, which imparts to the composition nonirritant properties, and which is capable of thickening the dissolved drug and facilitator to form a thickened composition as described above can be used. Typically, such compounds comprise thickening agents. The preferred thickening agent for use in the composition of the present invention is a hydrogenated vegetable oil which is available in various forms, for example, as a semi-solid that has the consistency of butter. In addition to its being a nonirritant and pharmaceutically acceptable, the hydrogenated vegetable oil is compatible and stable with other constituents that can be used in formulating the composition and compositions formulated therefrom have prolonged shelf-life, for example, many months. It has been observed also that the hydrogenated vegetable oil imparts soothing properties to the composition and that, in its applied form, the composition is breathable.

Examples of other thickening agents than can be used are: cellulosic thickening agents, for example, carboxymethylcellulose; and acrylic thickening agents, for example, carbomers, for example, non-linear polymers of acrylic acid cross-linked with a polyalkenyl polyether, and alkyl acrylates, for example, acrylic acid/alkyl methacrylate copolymer.

The amount of thickener comprising the composition will depend on various factors, including the nature of the other ingredients of the composition and the amounts thereof. The non-thickened composition comprises the solid drug and facilitator dissolved in organic solvent and is typically in the form of a non-shape-retaining runny liquid. The thickener is used in an amount that converts the liquid preferably to an ointment, as described above. It is believed that, for most application, this can be accomplished by the use of about 5 to about 90 wt. % of the thickening agent.

Optionally, the composition can include also one or more other materials which impart desired properties, including nonirritant properties to the composition. Such materials will comprise typically compounds of the type that are used as additives in pharmaceutical compositions. Examples of such materials are preservatives, pH controlling agents, stabilizers, surfactants, and emulsifiers. It should be understood that the composition of the present invention should be free of any material that would cause the composition to be irritating in use.

The composition can be applied to the skin as often as needed to achieve the desired results. The frequency of application will vary depending on various factors, for example, the nature of the composition and the involved condition. The application to the skin of the composition at a temperature above room temperature, for example, up to 40° C., can be used to increase delivery through the skin of the drug. In general, application of the composition one or more times a day will be suitable for treating many conditions and can be continued for as long as is required to obtain the desired results, for example, weeks or months or indefinitely. In the use of laser treatment to blanch PWS, it is recommended that the composition be applied to the wound comprising the photocoagulated blood vessels at a time prior to the time the blood vessels begin to reperfuse.

As discussed above, it is known to use laser irradiation for the treatment of a PWS birthmark for the purpose of blanching the birthmark; the type of treatment which involves the use of a pulsed dye laser (PDL) is approved by the Food and Drug Administration. In addition, cryogen spray cooling (CSC) is a known pretreatment to laser irradiation in order to prevent negative skin surface textural changes such as scarring, atrophy, induration and dyspigmentation, by reducing the skin temperature. The particular “laser” conditions used will vary depending on various factors including, for example, the nature of the birthmark and the recipient. Exemplary conditions of PDL irradiation are as follows: (a) the use of about 0.45 ms to about 1.5 ms pulses, typically using a wavelength of about 585 to about 595 nanometers; (b) irradiation dosages of about 10 to about 15 joules/square centimeter; (c) spot diameters for the PDL beam of about 7 to about 10 millimeters; and (d) total irradiation time of about 5 to about 15 min depending on the size of the birthmark.

After PDL blanching of the entire PWS birthmark, a topical pharmaceutical composition of the present invention is applied to the entire area that has been treated and blanched, for example, once daily for one month to prevent revascularization. Such treatment is effective in retaining or prolonging the “blanched” appearance of the PWS birthmark.

### EXAMPLES

The following example is illustrative of a composition of the present invention and the use of the composition in the treatment of a blanched PWS birthmark.

### Example No. 1

This example is illustrative of a topical rapamycin-containing composition of the present invention in the form of an ointment which includes the dermal penetration-facilitator pentadecalactone.

| Ingredient | Amount, wt. % |
|---|---|
| rapamycin | 1 |
| pentadecalactone | 8 |
| benzyl alcohol | 6 |
| (solvent) | |
| hydrogenated vegetable oil | 85 |
| 100% triglyceride | |
| (thickening agent) | |
| AarhusKarlshamn AB | |

A composition comprising the above constituents can be prepared in any suitable way to form an ointment. For example, the composition can be prepared from a two-part mixture. One part is prepared by adding, with stirring, the solid rapamycin into a container of benzyl alcohol at room temperature to form a clear solution. A second part mixture is prepared by warming the hydrogenated vegetable oil to about 60° C. to soften it and then adding, with stirring, the pentadecalactone to the oil. This part is then cooled to about 40° C. and added, with stirring, to the solution of rapamycin, which is then cooled to about −10° C. The resulting ointment can be spread on the surface of the epidermis which has been laser-treated.

### Example No. 2

This example is illustrative of a method of treatment of port wine stain (PWS) birthmarks on the face of a human male according to the present invention; it involves pulsed dye laser (PDL) destruction of the subsurface blood vessels of the hypervascularized dermal area followed by topical treatment with the composition of Example No. 1 to prevent revascularization.

In order to minimize pain during pulsed dye laser irradiation, a topical anesthetic cream (4% lidocaine) is placed onto the skin surface about 90 minutes prior to laser irradiation. Immediately prior to laser radiation, the area to be irradiated is subjected to cryogen spray cooling, comprising a 50 millisecond (ms) spurt of the liquid cryogen tetrafluoroethane. As liquid cryogen evaporates rapidly, the superficial skin temperature is reduced as a result of supplying the latent heat of vaporization. The reduced skin temperature prevents negative skin surface textural changes, for example, scarring and dyspigmentation. PDL irradiation is performed in about 0.45 ms to about 1.5 ms pulses using a wavelength of about 585 to about 595 nanometers (nm). Irradiation dosages are about 10 to about 15 joules/square centimeter (J/cm²). The spot diameters for the PDL beam are about 7 to about 10 millimeters (mm). Total irradiation time is about 5 to about 15 min depending on the size of the birthmark. Subjects and all personnel present are equipped with protective eyeglasses to shield their eyes during the laser treatment.

Immediate post laser treatment includes the application of an ice pack to the treated areas for the first two hours after laser exposure. Thereafter, ice cooling is applied to the treated area for ten minutes out of every hour for the next 24 waking hours. The treated birthmark is blanched.

After PDL treatment of the entire PWS birthmark, the ointment of Example No. 1 is applied topically to the entire treated and blanched area once daily for one month to prevent revascularization. Such treatment is effective in retaining the “blanched” appearance of the PWS birthmark.

1. A nonirritant, thickened pharmaceutical composition comprising: (A) drug which is normally solid and which has anti-angiogenesis properties and/or immunosuppressant properties; (B) a normally solid dermal penetration-facilitator for the drug; (C) a solvent which is capable of dissolving the drug and the facilitator; and (D) a thickening agent.

2. A pharmaceutical composition which comprises:
A) a drug which has anti-angiogenesis properties and/or immunosuppressant properties and which is normally a solid that is insoluble in water, but which is present in the composition in dissolved form and in a pharmaceutically effective amount;
B) a dermal penetration-facilitator which is normally a solid and which is present in the composition in dissolved form and in an amount which is effective, upon topical application of the composition to the outer surface of the skin of a recipient, in promoting the delivery of the dissolved drug to the dermal layer of the skin, with minimal systemic delivery of the dissolved drug into the blood stream of the recipient; and
C) a single solvent or more than one solvent which is capable of dissolving the drug and which is present in the composition in an amount sufficient to dissolve the drug and the facilitator.

3. A composition according to claim 1 which includes a Hsieh penetration-facilitator.

4. A composition according to claim 2 which includes a Hsieh penetration-facilitator.

5. A composition according to claim 3 which includes the drug rapamycin.

6. A composition according to claim 5 in which the penetration-facilitator is pentadecalactone.

7. A composition according to claim 4 which includes the drug rapamycin.

8. A composition according to claim 7 in which the penetration facilitator is pentadecalactone.

9. A composition according to claim 1, in which the solvent is benzyl alcohol.

10. A composition according to claim 2 in which the solvent is benzyl alcohol.

11. A composition according to claim 2 which includes a thickening agent.

12. A composition according to claim 11 wherein the thickening agent is hydrogenated vegetable oil.

13. A composition according to claim 1 which includes one or more of the following drugs: rapamycin (also known as sirolimus), tacrolimus, everolimus, gusperimus, pimecrolimus, tesirolimus, cyclosporine, and temsirolimus.

14. A composition according to claim 2 which includes one or more of the following drugs: rapamycin (also known as sirolimus), tacrolimus, everolimus, gusperimus, pimecrolimus, tesirolimus, cyclosporine, and temsirolimus.

15. A method for treating a bodily portion which is afflicted with psoriasis, Kaposi's sarcoma, a pre-cancer lesion, skin cancer or rosacea comprising applying to the bodily portion a composition according to claim 2.

16. In a method for treating a bodily portion which is associated with a port wine stain birthmark (PWS birthmark) in which bodily portion the flow of blood has been disrupted intentionally for the purpose of blanching the PWS birthmark and wherein the flow of blood in said bodily portion tends to be restored through the healing process of the body, the improvement comprising transdermally treating the bodily portion pharmaceutically in a nonirritant and non-systemic manner to inhibit the restoration of the flow of blood to the extent that the blanching of the PWS birthmark is prolonged.

17. In a method according to claim 16, the improvement in which the pharmaceutical treatment includes applying to said bodily portion a pharmaceutical composition which includes a drug having anti-angiogenesis and/or immunosuppressant properties.

18. A method according to claim 17 in which the disruption of said flow of blood has been effected by treating said birthmark with a pulsed dye laser for a period of time sufficient to blanch the birthmark.

19. A method according to claim 18 including applying cryogen spray cooling to said bodily portion prior to treating said birthmark with said laser.

20. A method according to claim 17 wherein the composition applied to said bodily portion is a composition according to claim 2.
